(19) United States
(12) Patent Application Publication
(10) Pub. No.: US 2026/0355071 A1
(43) Pub. Date: Nov. 12, 2026

(54) POWDER SOLID COSMETIC

(71) Applicant: SHISEIDO COMPANY, LTD., Tokyo (JP)

(72) Inventor: Tatsuya YONEMIZU, Tokyo (JP)

(73) Assignee: SHISEIDO COMPANY, LTD., Tokyo (JP)

(21) Appl. No.: 19/132,913

(22) PCT Filed: Dec. 6, 2023
(86) PCT No.: PCT/JP2023/043568

§ 371 Date: May 27, 2025

(30) Foreign Application Priority Data
Dec. 27, 2022 (JP) 2022-209735

(51) Int. Cl. A61K 8/02; A61K 8/25; A61Q 19/00

(52) U.S. Cl. CPC A61K 8/0254; A61K 8/025; A61K 8/25; A61Q 19/00; A61K 2800/10; A61K 2800/43; A61K 2800/622; A61K 2800/624; A61K 2800/651

(57) ABSTRACT

The present invention provides a powder solid cosmetic excellent in usability and moldability. A powder solid cosmetic according to the present invention includes: (A) a metal soap-treated plate-like powder; (B) an amino acid-treated plate-like powder; and (C) a non-elastic spherical powder. Further, the cosmetic may contain an oil component and a pigment.

### TECHNICAL FIELD

The present invention relates to a solid cosmetic. More specifically, the present invention relates to a powder solid cosmetic having excellent moldability and usability.

### BACKGROUND ART

Conventionally, powder solid cosmetics such as foundation generally contain talc and fine resin powder. This is because talc is effective for improving the moldability of the solid cosmetic, and fine resin powder is effective for imparting excellent usability to the cosmetic. However, talc is rich in irregularity shapes, and therefore moldability and impact resistance of a powder solid cosmetic can be improved. However, powder solid cosmetics containing talc had room for improvement in terms of smoothness of feel from the viewpoint of usability, and in that the appearance could become powdery when applied to the skin from the viewpoint of finish quality. For this reason, the development of cosmetics that do not contain talc or resin powders has been desired.

From such a viewpoint, it has been studied to use mica instead of talc, but it has been difficult to achieve sufficient performance from the viewpoint of improving moldability. For a fine resin powder, use of silica as a substitute has been studied, but usability is often insufficient or moldability is often insufficient.

From the background as described above, there has been a demand for a cosmetic that does not contain talc or the like and achieves excellent moldability and usability.

### CITATION LIST

### Patent Literature

Patent Literature 1: JP 2018-177640 A

### SUMMARY OF THE INVENTION

An object of the present invention is to provide a powder solid cosmetic capable of achieving excellent usability and moldability.

According to the present invention, the following invention is provided.

[1]

A powder solid cosmetic including:
(A) a metal soap-treated plate-like powder;
(B) an amino acid-treated plate-like powder; and
(C) a non-elastic spherical powder.
   [2]

The cosmetic according to [1], in which the component (A) is metal soap-treated mica.

[3]

The cosmetic according to [1] or [2], in which the component (B) is amino acid-treated mica.

[4]

The cosmetic according to any one of [1] to [3], in which the component (C) is an inorganic spherical powder or an organic spherical powder having a crushing strength per particle of 30 MPa or more.

[5]

The cosmetic according to any one of [1] to [4] in which the component (C) is a powder selected from the group consisting of spherical silica, spherical starch powder, spherical calcium carbonate, spherical glass, and spherical cellulose.

[6]

The cosmetic according to any one of [1] to [5], further including: (D) an oil component.

[7]

The cosmetic according to any one of [1] to [6], further including: (E) a colorant.

[8]

The cosmetic according to [7], in which the component (E) is (E1) an amino acid-treated pigment or a metal soap-treated pigment.

[9]

The cosmetic according to any one of [1] to [8], in which
a content of the component (A) is 20 to 50 mass %,
a content of the component (B) is 10 to 40 mass %, and
a content of the component (C) is 2 to 20 mass %,
based on a total mass of the cosmetic.
   [10]

The cosmetic according to any one of claims [1] to [89], in which a ratio B/A between a content amount of the component (B) and a content amount of the component (A) is 0.2 to 2.

[11]

The cosmetic according to any one of claims [1] to [10], in which a total content amount of the content amount of the component (A) and the content amount of the component (B) is 30 to 70 mass %, based on the total mass of the cosmetic.

[12]

The cosmetic according to any one of claims [1] to [11], in which a content of untreated talc and surface-treated talc is 0 to 5 mass %, based on the total mass of the cosmetic.

[13]

The cosmetic according to any one of claims [1] to [12], in which a content of the resin powder is 0 to 5 mass % based on the total mass of the cosmetic.

According to the present invention, there is provided a powder solid cosmetic that achieves excellent usability and moldability.

### BEST MODE FOR CARRYING OUT THE INVENTION

### [Powder Solid Cosmetic]

A powder solid cosmetic according to the present invention includes:
(A) a metal soap-treated plate-like powder;
(B) an amino acid-treated plate-like powder; and
(C) a non-elastic spherical powder. Each component contained in the cosmetic according to the present invention will be described below.

### [(A) Metal Soap-Treated Plate-Like Powder]

The powder solid cosmetic according to the present invention includes a metal soap-treated plate-like powder (hereinafter may be referred to as a component (A)). The metal soap-treated plate-like powder is obtained by subjecting a plate-like powder to a metal soap treatment.

The plate-like powder is a powder having an aspect ratio (average particle size/average thickness) of more than 1, for example, at least 5 or more. The aspect ratio is preferably 20 or more, more preferably 20 to 80, and still more preferably 25 to 60.

The powder can be selected from various inorganic materials. Specifically, the powder can be selected from mica, synthetic mica, synthetic phlogopite, sericite, kaolin, alumina, barium sulfate, boron nitride, zinc oxide, silica, fish scale foil, bismuth oxychloride, and the like. In consideration of the influence on the human body, it is preferable not to select talc.

In the present invention, a metal soap-treated plate-like powder obtained by subjecting these powders to metal soap treatment is used. The metal soap is a salt of a carboxyl group-containing compound such as a fatty acid and a divalent or higher valent metal. Here, the carboxyl group-containing compound constituting the metal soap includes, for example, a fatty acid having a linear or branched alkyl group, carboxyl-modified silicone, and the like, and among them, a higher fatty acid, that is, a linear or branched carboxylic acid having 8 to 24 carbon atoms, preferably 12 to 18 carbon atoms is preferable. Preferred specific examples of the higher fatty acid include stearic acid, isostearic acid, myristic acid, and lauric acid.

Examples of the divalent or higher valent metal constituting the metal soap preferably include, but are not limited to, alkaline earth metals such as calcium, magnesium, and zinc, and trivalent metals such as aluminum.

That is, examples of the metal soap preferably used as the surface treatment agent include magnesium myristate, zinc myristate, aluminum myristate, magnesium stearate, zinc stearate, aluminum stearate, aluminum distearate, aluminum oleate, aluminum palmitate, aluminum laurate, aluminum myristate, aluminum dimyristate, and silicone metal soaps (divalent or trivalent metal salts of organosiloxane derivatives having terminal carboxyl groups).

In the present invention, as the component (A), any plate-like powder treated with any metal soap can be used, and among them, metal soap-treated mica is preferable.

In the present invention, the content of the component (A) is not particularly limited, but the content of the component (A) is preferably 20 to 50 mass %, and more preferably 25 to 45 mass %, based on the total mass of the cosmetic composition.

### [(B) Amino Acid-Treated Plate-Like Powder]

The solid cosmetic according to the present invention includes an amino acid-treated plate-like powder (hereinafter may be referred to as a component (B)) in addition to the component (A). The amino acid-treated plate-like powder is obtained by subjecting a plate-like powder to an amino acid treatment.

As the plate-like powder, the plate-like powder exemplified as the component (A) can be used. In the present invention, an amino acid-treated plate-like powder obtained by subjecting these powders to an amino acid treatment is used, but amino acid-treated mica is preferable.

The amino acid used for the amino acid treatment can be randomly selected according to the purpose. In the amino acid-treated powder, the organic group of the amino acid is considered to be bonded to the powder surface via the nitrogen atom.

In the present invention, the amino acid is preferably one having an aliphatic group to be examined, and specifically, an N-mono-long chain aliphatic acyl basic amino acid or a salt thereof is preferable. More specific examples include N-lauroyllysine, N-myristyllysine, N-stearyllysine, N-capryllysine, N-behenyllysine, N-palmitoyllysine, and salts thereof. N-lauroyllysine or a salt thereof is preferable from the viewpoint of ease of handling. As the salts, metal salts such as aluminum, zinc, magnesium, calcium, germanium, titanium, and zirconium are preferable.

In the treatment of the plate-like powder, the blending ratio of the amino acid or the salt thereof is preferably 0.1 to 10 mass % based on the mass of the plate-like powder. When the content is less than 0.1 mass %, the water repellent effect by the surface treatment is not sufficient, and when the content exceeds 10 mass %, the effect by the surface treatment is saturated, which tends to be economically disadvantageous.

Next, as a method for treating the powder, either a dry method or a wet method may be used, but the water repellent effect by the surface treatment is higher in the wet method. The dry method is obtained by stirring and mixing or co-pulverizing a powder of an amino acid or a salt thereof having an average particle size of 0.1 to 5 μm and a plate-like powder. The wet method is obtained by bringing a plate-like powder into contact with a solution in which an amino acid or a salt thereof is dissolved in strongly acidic or strongly alkaline water, then neutralizing the solution to near neutral to deposit the amino acid or the salt thereof on the surface of the powder while precipitating the amino acid or the salt, washing and removing the salt generated by the neutralization step, and drying the solution.

In the present invention, the content of the component (B) is not particularly limited, but the content of the component (B) is preferably 10 to 40 mass %, and more preferably 10 to 35 mass %, based on the total mass of the cosmetics.

In addition, the cosmetic composition according to the present invention includes two types of plate-like powders of the component (A) and the component (B), and the use feeling and moldability are affected by the blending ratio of these. Therefore, the ratio B/A of the content amount of the component (B) to the content amount of the component (A) is preferably 0.5 to 2, and more preferably 0.6 to 1.2.

### [(C) Non-Elastic Spherical Powder]

The solid cosmetic according to the present invention further includes a non-elastic spherical powder (hereinafter may be referred to as a component (C)). The shape of the component (C) needs to be spherical, but does not need to be strictly true spherical, and may be, for example, spherical with an elliptical cross section. Specifically, the aspect ratio is preferably less than 5. When the cosmetic is used, the spherical shape is preferable from the viewpoint of obtaining a more smooth and preferable feel.

The component (C) is non-elastic, but specifically, the crushing strength per particle is preferably 30 MPa or more, more preferably 35 MPa or more, and still more preferably 40 MPa or more. The component (C) may be either an inorganic spherical powder or an organic spherical powder. The crushing strength per particle in the present invention is a value obtained by calculating the strength at the time when a load force is applied to one sample particle at a constant increasing rate using a micro compression tester MCT-211 manufactured by Shimadzu Corporation and the particle is broken. When the crushing strength is less than 30 MPa, the flexibility is too high to provide sufficient molding strength, and the dispersibility of the powder cannot be sufficiently improved.

The average particle size of the component (C) is preferably in the range of 1 to 30 μm, more preferably in the range of 3 to 20 μm, and still more preferably in the range of 5 to 15 μm. When the average particle size is less than 1 μm, the smoothness at the time of applying the cosmetic is lost, and when the average particle size is more than 30 μm, there is a tendency for a creaky feeling or a gritty foreign body sensation to occur.

Examples of the component (C) include spherical silica, spherical starch powder, spherical calcium carbonate, spherical glass, and spherical cellulose. In particular, spherical silica is preferable from the viewpoint of usability, and porous silica particles are more preferable. In addition, spherical glass is also preferably used because it can improve the usability and also improves the “scooping” of a powder solid cosmetic.

In the present invention, the content of the component (C) is not particularly limited, but the content of the component (C) is preferably 2 to 20 mass %, and more preferably 5 to 15 mass %, based on the total mass of the cosmetic composition.

### [(D) Oil Component]

The cosmetic composition according to the present invention preferably further contains an oil component (hereinafter may be referred to as a component (D)) in addition to the components (A) to (C). The oil component has an effect of improving the moldability of the powder solid cosmetic and also improving the usability.

The oil component that can be used in the present invention is not particularly limited as long as the oil component can be generally used. Specific examples thereof include liquid oil-and-fat, solid oil-and-fat, wax, hydrocarbon, higher fatty acid, higher alcohol, synthetic ester oil, and silicone oil.

In the present invention, the content of the component (D) is not particularly limited, but when the cosmetic composition according to the present invention contains the component (D), the content of the component (D) is preferably 1 to 30 mass %, and more preferably 2 to 10 mass %, based on the total mass of the cosmetic composition.

### [(E) Colorant]

The cosmetic composition according to the present invention preferably further contains a colorant (hereinafter may be referred to as the component (E)) in addition to the components (A) to (D). The colorant is useful when the cosmetic is intended to color the skin or the like.

The colorant can be randomly selected from those that can be generally used for cosmetics, but a pigment is preferable. Examples of the pigment include the following pigments:
organic pigment (for example, Red No. 104, Red No. 202, Red No. 226, Yellow No. 4, Yellow No. 5, Blue No. 1, Black No. 401, and the like);
lake pigment (for example, yellow No. 4 aluminum lake, blue No. 1 aluminum lake, and the like);
inorganic coloring pigment (for example, yellow iron oxide, red iron oxide, black iron oxide, chromium oxide, chromium hydroxide, carbon black, ultramarine blue, Prussian blue, manganese violet, and the like);
inorganic white pigment (for example, zinc oxide, titanium oxide (pigment grade), and the like);
extender pigment (for example, mica, sericite, and the like); and
other inorganic pigments (for example, barium sulfate, magnesium carbonate, aluminum silicate, magnesium silicate, alumina, and the like)

These pigments can be incorporated as such into the cosmetic according to the present invention, but more preferably, the pigments are amino acid-treated pigments or metal soap-treated pigments as described above. This is because the effect of the present invention is more remarkably exhibited when the pigment is treated in the same manner as the plate-like powder.

### [Other Components]

In the powder solid cosmetic according to the present invention, in addition to the components (A) to (E), if necessary, water, a surfactant, a pH adjusting agent, a moisturizing agent, a thickener, a dispersant, a stabilizer, an antiseptic agent, an antioxidant, a perfume, a dye, and the like can also be appropriately blended within the range of achieving the object of the present invention. In the present invention, the content of surface-treated talc such as untreated talc and metal soap-treated talc is preferably low. Specifically, preferably, the content of the untreated talc and the surface-treated talc is 0 to 5 mass % based on the total mass of the cosmetic, and particularly preferably, the untreated talc and the surface-treated talc are not contained at all. In addition, from the same viewpoint, preferably, the content amount of fine resin powder, so-called microplastic beads, is also small. Specifically, preferably, the content of fine resin particles is 0 to 5 mass % based on the total mass of the cosmetic, and particularly preferably, the fine resin particles are not contained at all.

As the product form of the powder solid cosmetic according to the present invention, it is possible to take any product form within the category of powder cosmetic. Specifically, product forms such as foundation, eye shadow, cheek color, body powder, perfume powder, baby powder, pressed powder, deodorant powder, and face powder.

### EXAMPLES

Hereinafter, the present invention will be described in more detail with reference to Examples, but the present invention is not limited thereto at all. The blending amount is shown in mass % with respect to the system in which the component is blended unless otherwise specified.

### Examples 1 to 6, Reference Example 1, and Comparative Examples 1 to 5

Powder solid cosmetics having compositions shown in Tables 1 and 2 below were produced, and evaluated for the following items. The results are also shown in Tables 1 and 2.

### Usability and Finish

Each produced powder cosmetic was applied to the face by 10 expert panels, and the usability (spreadability on the skin, smoothness, and lack of powderiness) and finish (uniformity and lack of gloss) at the time of application were evaluated. The evaluation criteria are as follows.
A: 8 or more of 10 panelists answered that the usability and finish were good
B: 5 or more and 7 or less of 10 panelists answered that the usability and finish were good.
C: 4 or less of 10 panelists answered that the usability and finish were good.

### Moldability (Impact Resistance)

Each of the produced powder solid cosmetics was placed in a dedicated compact case and dropped from a certain height for evaluation. The molded product was dropped and evaluated by the number of times until the molded product was broken, and the evaluation criteria are as follows.
A: 7 times or more
B: 5 times or more and less than 7 times
C: less than 5 times

When the number of drops until cracking was 5 or more, it was determined that good impact stability was exhibited.

TABLE 1

| | | | Reference | Comparative | | |
|---|---|---|---|---|---|---|
| | | Example | Example | Example | | |
| Component | | 1 | 1 | 1 | 2 | 3 |
| (A) Magnesium stearate-treated mica | | 25 | — | — | — | 25 |
| (B) Lauroyllysine-treated mica | | 20 | — | — | 20 | — |
| Talc | | — | balance | — | — | — |
| Calcium stearate-treated talc | | — | 15 | — | — | — |
| Silicone-treated mica | | — | 25 | — | — | — |
| Synthetic mica | | balance | — | balance | balance | balance |
| (C) Silica (average particle | | 5 | — | 5 | 5 | 5 |
| size: 10 μm) | | | | | | |
| (C) Silica (average particle | | 5 | — | 5 | 5 | 5 |
| size: 7~12 μm) | | | | | | |
| Calcium/aluminum borosilicate | | 5 | 5 | 5 | 5 | 5 |
| Polymethylsilsesquioxane | | — | 5 | — | — | — |
| Methyl methacrylate crosspolymer | | — | 10 | — | — | — |
| Zinc oxide | | 3 | 3 | 3 | 3 | 3 |
| Zinc myristate | | 3 | 3 | 3 | 3 | 3 |
| Petrolatum | | 0.5 | 0.5 | 0.5 | 0.5 | 0.5 |
| Sorbitan sesquiisostearate | | 1 | 1 | 1 | 1 | 1 |
| Triethylhexanoin | | 3.5 | 3.5 | 3.5 | 3.5 | 3.5 |
| Diisostearyl malate | | 1.5 | 1.5 | 1.5 | 1.5 | 1.5 |
| Iron oxide | | — | 0.5 | — | — | — |
| Sodium dilauramidoglutamide | | 0.5 | — | 0.5 | 0.5 | 0.5 |
| lysine-treated iron oxide | | | | | | |
| Chlorphenesin | | q.s. | q.s. | q.s. | q.s. | q.s. |
| Tocopherol | | q.s. | q.s. | q.s. | q.s. | q.s. |
| Total amount | | 100 | 100 | 100 | 100 | 100 |
| B/A | | 0.8 | — | — | — | — |
| Usability | Spreadability on the skin | A | B | B | B | B |
| | Smoothness | A | A | C | B | A |
| | Lack of powderiness | A | B | C | B | B |
| Finish | Uniformity | A | B | C | C | B |
| | lack of gloss | A | A | B | B | B |
| Drop test | Average of N = 3 trials | 9.00 | 12.00 | 3.33 | 4.00 | 3.67 |
| | Evaluation | A | A | C | C | C |

TABLE 2

| | | Comparative | | | | | | Comparative |
|---|---|---|---|---|---|---|---|---|
| | | Example | Example | | | | | Example |
| Component | | 4 | 2 | 3 | 4 | 5 | 6 | 5 |
| (A) Magnesium stearate-treated mica | | 60 | 48 | 36 | 30 | 24 | 12 | — |
| (B) Lauroyllysine-treated mica | | — | 12 | 24 | 30 | 36 | 48 | 60 |
| Synthetic mica | | balance | balance | balance | balance | balance | balance | balance |
| (C) Silica (average particle | | 5 | 5 | 5 | 5 | 5 | 5 | 5 |
| size: 10 μm) | | | | | | | | |
| (C) Silica (average particle | | 5 | 5 | 5 | 5 | 5 | 5 | 5 |
| size: 7~12 μm) | | | | | | | | |
| Calcium/aluminum borosilicate | | 5 | 5 | 5 | 5 | 5 | 5 | 5 |
| Zinc oxide | | 3 | 3 | 3 | 3 | 3 | 3 | 3 |
| Zinc myristate | | 3 | 3 | 3 | 3 | 3 | 3 | 3 |
| Petrolatum | | 0.5 | 0.5 | 0.5 | 0.5 | 0.5 | 0.5 | 0.5 |
| Sorbitan sesquiisostearate | | 1 | 1 | 1 | 1 | 1 | 1 | 1 |
| Triethylhexanoin | | 3.5 | 3.5 | 3.5 | 3.5 | 3.5 | 3.5 | 3.5 |
| Diisostearyl malate | | 1.5 | 1.5 | 1.5 | 1.5 | 1.5 | 1.5 | 1.5 |
| Iron oxide | | — | 0.5 | — | — | — | — | — |
| Sodium dilauramidoglutamide | | 0.5 | — | 0.5 | 0.5 | 0.5 | 0.5 | 0.5 |
| lysine-treated iron oxide | | | | | | | | |
| Chlorphenesin | | q.s. | q.s. | q.s. | q.s. | q.s. | q.s. | q.s. |
| Tocopherol | | q.s. | q.s. | q.s. | q.s. | q.s. | q.s. | q.s. |
| Total amount | | 100 | 100 | 100 | 100 | 100 | 100 | 100 |
| B/A | | — | 0.25 | 0.67 | 1 | 1.5 | 4 | — |
| Usability | Spreadability on the skin | A | A | A | A | B | B | B |
| | Smoothness | A | A | A | A | A | B | B |
| | Lack of powderiness | A | A | A | A | B | B | C |
| Finish | Uniformity | A | A | A | A | B | B | C |
| | lack of gloss | A | A | A | A | B | B | B |
| Drop test | Average of N = 3 trials | 3.67 | 10.33 | 9.33 | 8.33 | 7.67 | 6.33 | 4.33 |
| | Evaluation | C | A | A | A | A | B | C |

1. A powder solid cosmetic comprising:
(A) a metal soap-treated plate-like powder;
(B) an amino acid-treated plate-like powder; and
(C) a non-elastic spherical powder.

2. The cosmetic according to claim 1, wherein the component (A) is metal soap-treated mica.

3. The cosmetic according to claim 1, wherein the component (B) is amino acid-treated mica.

4. The cosmetic according to claim 1, wherein the component (C) is an inorganic spherical powder or an organic spherical powder having a crushing strength per particle of 30 MPa or more.

5. The cosmetic according to claim 1, wherein the component (C) is a powder selected from the group consisting of spherical silica, spherical starch powder, spherical calcium carbonate, spherical glass, and spherical cellulose.

6. The cosmetic according to claim 1, further comprising: (D) an oil component.

7. The cosmetic according to claim 1, further comprising: (E) a colorant.

8. The cosmetic according to claim 7, wherein the component (E) is (E1) an amino acid-treated pigment or a metal soap-treated pigment.

9. The cosmetic according to claim 1, wherein
a content of the component (A) is 20 to 50 mass %,
a content of the component (B) is 10 to 40 mass %, and
a content of the component (C) is 2 to 20 mass %,
based on a total mass of the cosmetic.

10. The cosmetic according to claim 1, wherein a ratio B/A between a content amount of the component (B) and a content amount of the component (A) is 0.2 to 2.

11. The cosmetic according to claim 1, wherein a total content amount of the content amount of the component (A) and the content amount of the component (B) is 30 to 70 mass %, based on the total mass of the cosmetic.

12. The cosmetic according to claim 1, wherein a content of untreated talc and surface-treated talc is 0 to 5 mass %, based on the total mass of the cosmetic.

13. The cosmetic according to claim 1, wherein a content of the resin powder is 0 to 5 mass % based on the total mass of the cosmetic.
